(19) United States
(12) Patent Application Publication
(10) Pub. No.: US 2007/0141146 A1
(43) Pub. Date: Jun. 21, 2007

(54) Novel compositions

(75) Inventors: Vincenzo Re, Essex (GB); Laurence Robinson, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM PHARMCO PUERTO RICO INCORPORATE, San Juan, PR (US)

(21) Appl. No.: 10/576,403

(22) PCT Filed: Oct. 21, 2004
(86) PCT No.: PCT/GB04/04482

§ 371 Date: Nov. 10, 2006

(30) Foreign Application Priority Data
Oct. 21, 2003 (GB) 0324574.3

(51) Int. Cl. A61K 31/4439; A61K 9/22

(52) U.S. Cl. 424/468; 514/342

(57) ABSTRACT

An oral dosage form that provides controlled release of an active pharmaceutical agent, 5 -[4-[2-(N-methyl-N-(2 pyridyl)amino)ethoxy]benzyl]thiazolidine-2,4-dione (hereinafter ‘Compound A’) or a pharmaceutically acceptable salt or solvate thereof in different body environments, a process for the preparation of such an oral dosage form, and the use of such a dosage form in medicine.

### FIELD OF THE INVENTION

The present invention relates to an oral dosage form that provides controlled release of an active pharmaceutical agent, 5-[4-[2-(N-methyl-N-(2 pyridyl)amino)ethoxy]benzyl]thiazolidine-2,4-dione (hereinafter ‘Compound A’) or a pharmaceutically acceptable salt or solvate thereof in different body environments, to a process for the preparation of such an oral dosage form, and to the use of such a dosage form in medicine.

### BACKGROUND TO THE INVENTION

A controlled release formulation of pharmaceutically active compound, such as Compound A or a pharmaceutically acceptable salt or solvate thereof, which is designed to release the active compound over the course of several hours and which is administered orally must typically be able to release the active compound in more than one pH environment. For example, after about 2 hours on average the oral dosage form will pass from the patient's stomach at a pH of 1.5-2 to the patient's intestines with pH ranging from 5.5-7. Since it is unpredictable exactly how long the dosage form will remain in the stomach or the intestines, it is desirable that the release rate of the pharmaceutically active compound is similar under all of the pH conditions that will be experienced upon administration.

European Patent Application, Publication Number 0,306,228 relates to certain thiazolidinedione derivatives disclosed as having hypoglycaemic and hypolipidaemic activity. The compound of Example 30 of EP 0,306,228 is 5-[4-[2-(N-methyl-N-(2-pyridyl)amino)ethoxy]benzyl]thiazolidine-2,4-dione. International Patent Application, Publication Number WO 94/05659 discloses certain salts of the compounds of EP 0,306,228. The preferred salt of WO 94/05659 is the maleic acid salt at Example 1 thereof.

Compound A and its pharmaceutically acceptable salts and solvates have useful pharmaceutical properties. In particular, Compound A or a pharmaceutically acceptable salt or solvate thereof is indicated to be useful for the treatment and/or prophylaxis of diabetes mellitus, conditions associated with diabetes mellitus and certain complications thereof; osteoporosis, Alzheimer's Disease, psoriasis, asthma and metabolic syndrome.

Compound A and its pharmaceutically acceptable salts and solvates, especially the maleate salt, exhibit a marked pH dependent solubility i.e. they are more soluble at pH 2 (˜5 mg/ml) in the acidic regions associated with the stomach compared to their solubility in the near neutral pHs of the small intestine, pH 7 (˜0.08 mg/ml). The pH dependent solubility and potential rapid release in the stomach of Compound A causes difficulties in the formulation or oral dosage forms. It is desirable that release is controlled to take place over a period of hours. Such a formulation would require dosing only once a day, and this is likely to improve patient compliance.

International Patent Application, Publication Number WO 00/28990 describes various modified release pharmaceutical compositions comprising insulin sensitizers, including Compound A and pharmaceutically acceptable salts or solvates thereof.

International Patent Application, Publication Number WO 00/28990 describes a method of treating Type 2 diabetes mellitus and conditions associated with diabetes mellitus, using certain pharmaceutical compositions, including modified release compositions, which provide a Threshold Plasma Concentration of Compound A or a pharmaceutically acceptable salt or solvate thereof.

International Patent Application, Publication Number WO 03/068195 describes a controlled release oral dosage form comprising a pharmaceutically active weak base such as Compound A or a pharmaceutically acceptable salt or solvate thereof.

It is an object of the present invention to provide an oral dosage form comprising a Compound A or a pharmaceutically acceptable salt or solvate thereof, which provides a maximized beneficial effect on glycaemic control for an extended period of time. Such a dosage form is considered to be suitable for once daily administration.

The present invention is based on the finding that certain glyceride based materials can be used as a matrix for oral dosage forms containing Compound A, and the resultant dosage forms have advantageous controlled release properties in the different pH conditions experienced by an oral dosage form after swallowing (i.e. upon administration).

Bodmeier et al (Drug Development and Industrial Pharmacy, 1990, 16(9), 15015-1519) have described the use of blends of waxes having different HLB values to control the release of propranolol HCl and theophylline, in a manner that is independent of the pH of the dissolution medium. Such formulations are formed by the heating and subsequent cooling of hard gelatin capsules filled with drug-wax powder blends.

### SUMMARY OF THE INVENTION

In one aspect the present invention provides a controlled release oral dosage form comprising Compound A, or a pharmaceutically acceptable salt or solvate thereof, dispersed in a carrier comprising a pharmaceutically acceptable waxy mixture of glyceride-based materials having a range of HLB values of 4 to 12 (preferably from 6 to 8), and an average melting point in the range of 50 to 55° C.

For the avoidance of doubt, as used herein the term “HLB value” shall mean hydrophilic-lipophilic balance. HLB values may be measured in accordance with the methods described in A. Gennaro and J. Remington, Remington's Pharmaceutical Sciences, 18th Edition, Mack Publishing Company, Easton, 1990, 304 and W. C. Griffith, J. Soc. Cosmetic Chemists, 1949, 1, 311.

As used herein, the term “controlled release” means a composition which has been designed to produce a desired pharmacokinetic profile by choice of formulation. For example, the term “controlled release” shall comprise delayed, pulsed and sustained release either alone or in any combination, suitably a sustained release. Controlled release also includes controlled release compositions in combination with non-controlled release compositions.

In another aspect the present invention provides a controlled release oral dosage form comprising Compound A, or a pharmaceutically acceptable salt or solvate thereof, dispersed in a carrier comprising a mixture of:
(a) a pharmaceutically acceptable waxy mixture of glyceride-based materials having an HLB value of greater than 12 (preferably greater than 8) and an average melting point in the range of 50 to 55° C., and an amount of
(b) a pharmaceutically acceptable fatty acid glyceride or glyceride mixture having an HLB value less than the HLB value of component (a) and an average melting point in the range of 50 to 55° C.,
   such that the carrier as a whole has an HLB value of 4 to 12 (preferably between 6 and 8).

In a further aspect, the present invention provides a controlled release oral dosage form comprising a first composition and a second composition, each composition comprising Compound A or a pharmaceutically acceptable salt or solvate thereof and a pharmaceutically acceptable carrier therefore, wherein:
(a) the carrier of the first composition comprises a pharmaceutically acceptable waxy mixture of glyceride-based materials having a range of HLB values of 4 to 12 (preferably from 6 to 8), and an average melting point in the range of 50 to 55° C.; and
(b) the carrier of the second composition comprises one or more pharmaceutically acceptable glyceride-based materials having a higher HLB value and/or lower average melting point than the carrier of the first composition.

Suitably, the second composition comprises one or more pharmaceutically acceptable glyceride-based materials having a higher HLB value and a lower average melting point than the carrier of the first composition.

It will be appreciated that the release rate of the drug from the first and second compositions is dependent upon the nature of pharmaceutically acceptable carrier(s) employed. Thus, different release rates may be achieved by the use of different glyceride-based materials in each of the said first and second compositions.

Suitably, the first and second compositions are arranged to release Compound A or a pharmaceutically acceptable salt or solvate thereof (‘the drug’), at differing release rates on administration such that the rate of release of the drug from the dosage form is substantially independent of pH.

Suitably, the release rate of the drug from the second composition is substantially greater than from the first composition. It is envisaged that, the second composition is an immediate release composition. It is also envisaged that, the first composition is a controlled release composition, such as a sustained release composition.

In one aspect, the second composition is arranged so that in use it releases substantially all of the drug in the stomach.

In a further aspect, the first composition is arranged so that in use it releases substantially all of the drug in the small intestine.

In one embodiment of the invention the carrier consists essentially of a pharmaceutically acceptable waxy mixture of glyceride-based materials having a range of HLB values of 4 to 12 (preferably from 6 to 8), and an average melting point in the range of 50 to 55° C.

Typical oral dosage forms include swallow tablets and capsules.

It is an advantage of the carriers indicated above that they are waxy materials that are molten at elevated (i.e. above ambient) temperatures, and can therefore be moulded into tablets containing Compound A, that retain their structural integrity under normal handling conditions without the need for additional tabletting excipients. However it is desirable to apply a conventional soluble film coat on the tablet surface to prevent spoilage when handled by a patient. Also the molten waxy material can be filled into capsule shells, to form swallow capsules containing Compound A.

In all the dosage forms of this invention, the waxy matrix provides advantageous controlled release properties in the different pH conditions to which the matrix is exposed after swallowing. In particular, the release rate of Compound A at acid pHs associated with the stomach pH, is not significantly different from the release rate in the near neutral pH of the small intestine.

It will be appreciated that Compound A is a one of many known examples of pharmaceutically acceptable weak bases. As such, it is anticipated that the dosage form of the present invention may be used to administer as a vehicle for other pharmaceutically acceptable weak bases having similar physicochemical properties to Compound A, such as other weak bases.

As used herein the term “weak base” shall mean any base the conjugate acid of which has a pKa of less than 11.5; in accordance with The Pharmaceutical Handbook, 19th Edition, 1980, page 232. The term “pharmaceutically acceptable weak base” shall be interpreted accordingly. Suitable pharmaceutically acceptable weak bases or pharmaceutically acceptable salts or solvates thereof for use in the present invention include those compounds that exhibit a marked pH dependent solubility. Preferred pharmaceutically acceptable weak bases or pharmaceutically acceptable salts or solvates thereof for use in the present invention are more soluble in the pH range from 1 to 3 than in the pH range from 4.5 to 8, i.e. they are more soluble in the acidic conditions found in the mammalian stomach than in the near neutral conditions of the mammalian intestines.

### BRIEF DESCRIPTION OF THE DRAWINGS

FIG. 1 is a graph of dissolution against time for two oral dosage forms in accordance with this invention, as disclosed in Examples 1 and 2.

FIG. 2 shows dissolution variability observed for cured and uncured oral dosage forms stored at 25° C./60% RH.

FIG. 3 shows physical stability observed for cured and uncured oral dosage forms stored at 25° C./60% RH.

FIG. 4 shows dissolution rate for the formulation of Example 3.

### DETAILED DESCRIPTION OF THE INVENTION

The pharmaceutically acceptable waxy mixture of glyceride-based materials is suitably a pharmaceutically acceptable glyceride-based waxy material obtainable by an alcoholysis/esterification reaction between a vegetable oil and a polyethylene glycol.

In the above indicated alcoholysis/esterification reaction, the vegetable oil is preferably a hydrogenated oil so that the fatty acid components are saturated. The reaction between a hydrogenated vegetable oil and a polyethylene glycol results in a mixture of fatty acid mono-, di-, and tri-glycerides and mono-and di-fatty acid esters of polyethylene glycol.

For a controlled release composition, the vegetable oil is preferably selected so that the predominant fatty acids are palmitic and stearic acids (C16 and C18 acids). A suitable oil is hydrogenated palm oil.

For an immediate release composition, the vegetable oil is preferably selected so that the predominant fatty acid is lauric acid (C12 acid). A suitable oil is hydrogenated palm oil.

The polyethylene glycol (or PEG) may have a mean molecular weight value ranging from 1300 to 1700. Preferably PEG 1500 is used.

Suitable waxy materials are those indicated as stearoyl macrogol glycerides in the European Pharmacopoeia, the relevant extracts of which are incorporated herein by reference thereto. Stearoyl macrogolglycerides are mixtures of monoesters, diesters, and triesters of glycerol, and monoesters and diesters of macrogols with a mean molecular mass between 300 and 4000 (nominal value).

As mentioned above, in order to make use of materials already approved as safe for human use, it may be necessary to use a mixture of materials whose properties approximate to a stearoyl macrogol glyceride having a range of HLB values of 4 to 12 (preferably between 6 and 8). For example it may be appropriate to use a stearoyl macrogol glyceride having a higher HLB value and blend it with another glyceride of more hydrophobic character.

A stearoyl macrogol glyceride that is suitable for human use and in which the fatty acid components are predominantly palmitic and stearic acids is available from Gattefosse as Gelucire® 50/13. This is described by the manufacturer as a stearoyl macrogol-32 glyceride which is synthesized by an alcoholysis/esterification reaction using hydrogenated palm oil and PEG 1500 as starting materials. It is therefore a well defined mixture of mono-,di-and triglycerides and mono-and di-fatty acid esters of polyethylene glycol. The predominant fatty acid is palmitostearic acid (C16-C18). It has a melting point in the range 46-51° C. and an HLB value of 13.

A stearoyl macrogol glyceride that is suitable for human use and in which the fatty acid components is predominantly lauric acid is available from Gattefosse as Gelucire® 44/14. This is described by the manufacturer as a lauroyl macrogol glyceride which is synthesized by an alcoholysis/esterification reaction using hydrogenated palm oil and PEG 1500 as starting materials. It is therefore a well defined mixture of mono-,di-and triglycerides and mono-and di-fatty acid esters of polyethylene glycol. The predominant fatty acid is lauric acid (C12). It has a melting point in the range 42.5-47.5° C. and an HLB value of 14.

A suitable additional component used in admixture to reduce the average HLB value to the desired range of 4 to 12 (preferably 6 to 8) may be, for example, a fatty acid glyceride mixture, also preferably with palmitic and stearic acids predominating.

A fatty acid glyceride mixture with suitable properties is Gelucire® 50/02 from Gattefosse which has an average melting point of 50° C. and an HLB value of 2. Another fatty acid glyceride mixture with suitable properties is Precirol® ATO 5, also from Gattefosse, which has an average melting point of 55° C. and an HLB value of 2. It is synthesized by esterification of glycerol by palmitostearic acid (C16-C18 fatty acid). The raw materials used are of strictly vegetable origin and the reaction process involves no catalyst. The manufacturer indicates that Precirol® ATO 5 is composed of mono-, di and triglycerides of palmitostearic acid, the diester fraction being predominant.

An advantage of the blended carrier is that the proportions of the components can be varied to change the release profile of the carrier i.e. the rate of release can be reduced by increasing the amount of the more hydrophobic component (the component with the lower HLB value). For example, a suitable carrier may be prepared by blending Gelucire® 50/13 and Precirol® ATO 5 in proportions ranging from 40 to 70% of Precirol ATO5.

Also the release rate can be slowed by incorporating a given unit dose in a larger oral dosage form i.e. increasing the weight of carrier relative to a given weight of active compound.

TABLE 1
Effect of Precirol ATO5 concentration and
tablet weight on dissolution profile

| Dissolution Timepoint | 50% Precirol ATO5 - | 60% Precirol ATO5 - |
|---|---|---|
| (Hours) | 269 mg tablet weight | 400 mg tablet weight |
| 1 | 18 | 15 |
| 2 | 28 | 24 |
| 4 | 41 | 36 |
| 6 | 47 | 39 |
| 8 | 52 | 42 |
| 10 | 58 | 44 |
| 12 | 64 | 47 |
| 16 | 70 | 54 |

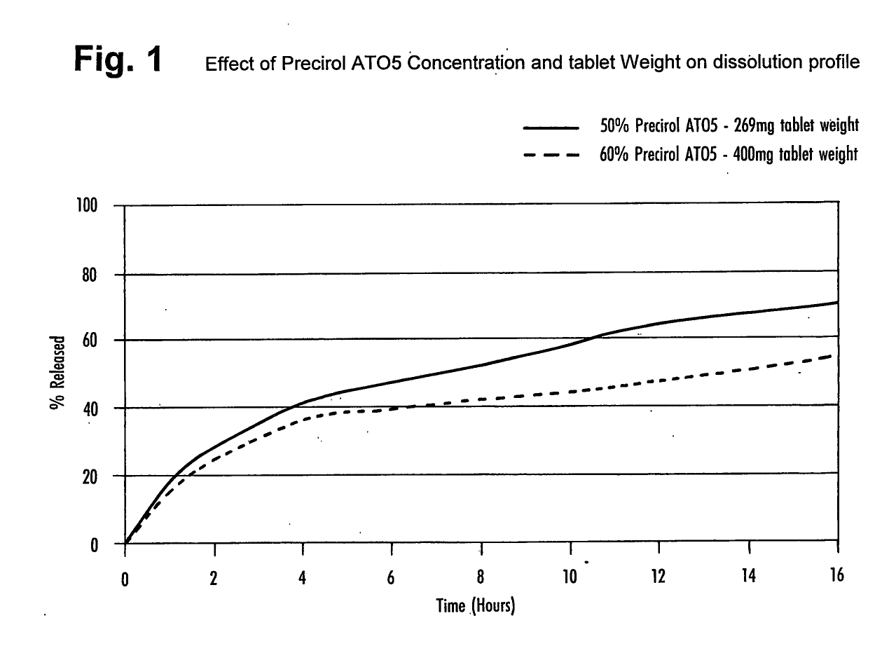

The above table of data is plotted on FIG. 1.

Swallow tablets of this invention are conveniently prepared by melting the waxy material, or melt-blending two materials when used, and dispersing Compound A or its salt or solvate in the molten wax. The molten blend is then filled in to moulds and allowed to solidify. The tablets thus formed are preferably provided with a film coat. Suitable coating agents include hydroxypropyl methylcellulose aqueous dispersions (which may include lactose or polydextrose), or preferably polyvinyl alcohol aqueous dispersions.

Swallow capsules of this invention are conveniently prepared by melting the waxy material, or melt-blending two materials when used, and dispersing Compound A or its salt or solvate in the molten wax. The molten blend is then filled in to capsule shells, such as hard gelatin capsule shells, in conventional manner.

Where the oral dosage form of the invention comprises a first and a second composition, each of the said compositions may be prepared independently using the aforementioned procedures. The resulting molten blend compositions may then be filled into moulds or capsules as desired. Suitably, the first and second compositions are arranged as discrete layers (e.g. as a bilayer).

Certain macrogol glycerides exist in more than one polymorphic form. We have found that is advantageous to heat treat the oral dosage forms after moulding and coating tablets, or filling capsules, by heating at a temperature below the melting point of the carrier, to convert the macrogol glyceride to its most stable form. Surprisingly this also results in a significant reduction in variability of the dissolution profile between individual oral dosage forms, which is a great advantage in accurate dosing. Heat treatment preferably takes place at about 40° C., for between 16 and 72 hours, for example, for 16, 24, 48 or 72 hours.

TABLE 2
Dissolution variability between cured and uncured
oral dosage forms stored at 25° C./60% RH.

| | Initial | 3 Months | |
|---|---|---|---|
| | immediately after | Curing at 40° C. | 3 Months |
| Dissolution | Curing | for 48 hours | No Curing |
| Timepoint | Max-Min Range between | | |
| (Hours) | n = 6 dosage forms (% dissolved) | | |
| 8 | 13 | 3 | 20 |
| 12 | 14 | 2 | 32 |
| 16 | 14 | 2 | 48 |

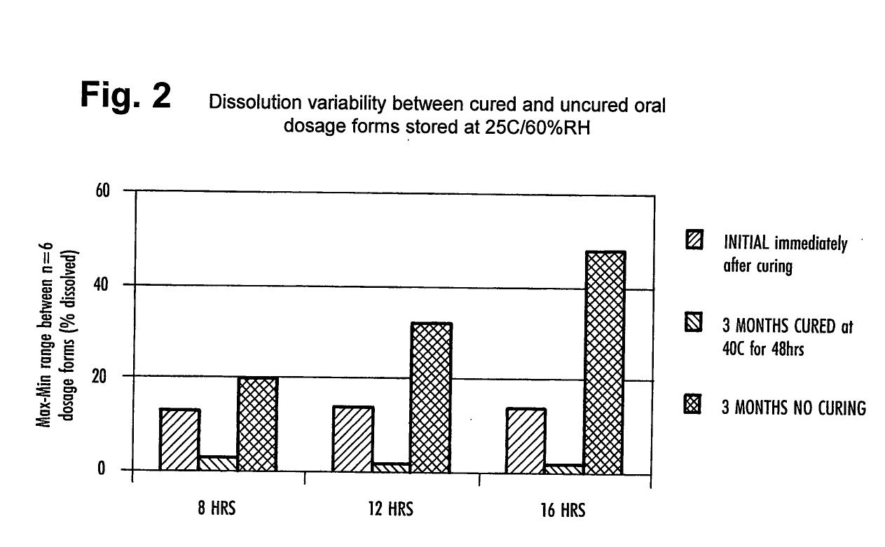

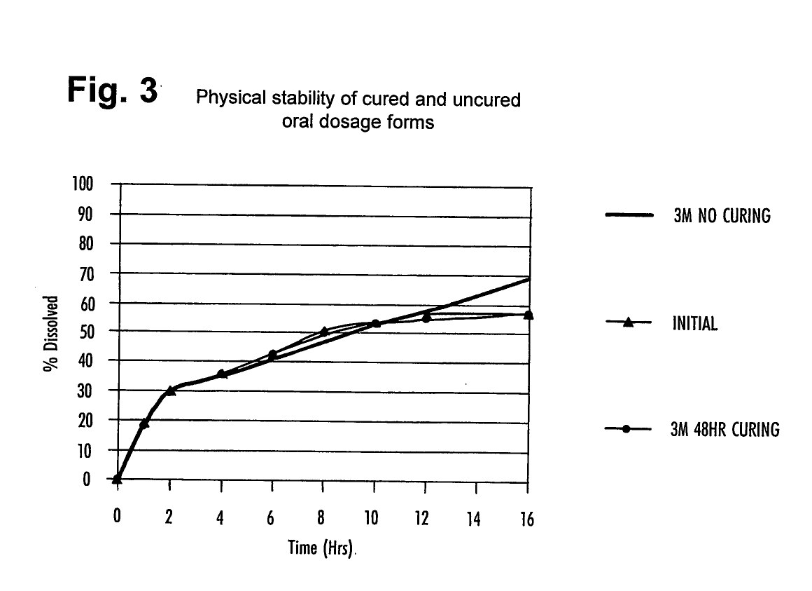

FIG. 2 and FIG. 3 show dissolution variability and physical stability observed for cured and uncured oral dosage forms stored at 25° C./60% RH.

Accordingly in a further aspect, the present invention provides a method of preparing a controlled release oral dosage form, which comprises dispersing Compound A, or a pharmaceutically acceptable salt or solvate thereof, in a molten carrier comprising a pharmaceutically acceptable waxy mixture of glyceride-based materials, having an HLB value of 4 to 12, and an average melting point in the range of 50 to 55° C., filling the molten mixture into tablet moulds or capsule shells, allowing the carrier to solidify, and optionally thereafter maintaining the solidified dosage form at a temperature of at least 40° C., but below the melting point of the carrier, for a time sufficient to allow the carrier to achieve a stable polymorphic form.

The invention also provides a method of preparing a controlled release oral dosage form, which comprises dispersing Compound A, or a pharmaceutically acceptable salt or solvate thereof, in a molten carrier comprising a mixture of
(a) a pharmaceutically acceptable waxy mixture of glyceride-based materials having an HLB value of greater than 12 and an average melting point in the range of 50 to 55° C., and an amount of
(b) a pharmaceutically acceptable fatty acid glyceride or glyceride mixture having an HLB value less than the HLB value of component (a) and an average melting point in the range of 50 to 55° C.,
   such that the carrier as a whole has an HLB value of 4 to 12,
   filling the molten mixture into tablet moulds or capsule shells, allowing the carrier to solidify, and optionally thereafter maintaining the solidified dosage form at a temperature of at least 40° C., but below the melting point of the carrier, for a time sufficient to allow the carrier to achieve a stable polymorphic form.

As mentioned above, Compound A, and its pharmaceutically acceptable salts and solvates have useful pharmaceutical properties. In particular it is indicated to be useful for the treatment and/or prophylaxis of diabetes mellitus, conditions associated with diabetes mellitus and certain complications thereof; osteoporosis, Alzheimer's Disease, psoriasis, asthma and metabolic syndrome (hereinafter referred to as the “Disorders of the Invention”). Suitably, Compound A or a pharmaceutically acceptable salt or solvate thereof when administered in an oral dosage form of this invention is indicated to be useful in the treatment and/or prophylaxis of diabetes mellitus, conditions associated with diabetes mellitus and certain complications thereof. Suitably, Compound A or a pharmaceutically acceptable salt or solvate thereof when administered in an oral dosage form of this invention is indicated to be useful in the treatment and/or prophylaxis of osteoporosis. Suitably, Compound A or a pharmaceutically acceptable salt or solvate thereof when administered in an oral dosage form of this invention is indicated to be useful in the treatment and/or prophylaxis of Alzheimer's Disease. Suitably, Compound A or a pharmaceutically acceptable salt or solvate thereof when administered in an oral dosage form of this invention is indicated to be useful in the treatment and/or prophylaxis of psoriasis. Suitably, Compound A or a pharmaceutically acceptable salt or solvate thereof is indicated to be useful in the treatment and/or prophylaxis of asthma. Suitably, Compound A or a pharmaceutically acceptable salt or solvate thereof when administered in an oral dosage form of this invention is indicated to be useful in the treatment and/or prophylaxis of metabolic syndrome.

The term ‘prophylaxis of conditions associated with diabetes mellitus’ includes the treatment of conditions such as insulin resistance, impaired glucose tolerance, impaired fasting glucose, hyperinsulinaemia and gestational diabetes. Diabetes mellitus preferably means Type II diabetes mellitus.

Conditions associated with diabetes include hyperglycaemia and insulin resistance and obesity. Further conditions associated with diabetes include hypertension, cardiovascular disease, especially Atherosclerosis, certain eating disorders, in particular the regulation of appetite and food intake in subjects suffering from disorders associated with under-eating, such as anorexia nervosa, and disorders associated with over-eating, such as obesity and anorexia bulimia Additional conditions associated with diabetes include polycystic ovarian syndrome and steroid induced insulin resistance.

The complications of conditions associated with diabetes mellitus encompassed herein includes renal disease, especially renal disease associated with the development of Type II diabetes including diabetic nephropathy, glomerulonephritis, glomerular sclerosis, nephrotic syndrome, hypertensive nephrosclerosis and end stage renal disease.

Compositions of the present invention comprising Compound A are also indicated to be useful in the treatment and/or prohylaxis of certain other conditions in which agonism of the PPAR-γ receptor pathway is beneficial.

In a preferred embodiment the present invention provides a method for the treatment and/or prophylaxis of the Disorders of the Invention which method comprises administering an oral dosage form of this invention comprising Compound A or a pharmaceutically acceptable salt or solvate thereof, to a human or non-human mammal in need thereof.

In a further preferred embodiment the present invention provides an oral dosage form of the invention comprising Compound A or a pharmaceutically acceptable salt or solvate thereof for use in the treatment and/or prophylaxis of the Disorders of the Invention.

As used herein, the term “pharmaceutically acceptable” embraces compounds, compositions and ingredients for both human and veterinary use. For example the term “pharmaceutically acceptable salt” embraces a veterinarily acceptable salt. In particular, suitable pharmaceutically acceptable salted forms of Compound A include those described in European Patent Number 0 306 228 and International Patent Application, Publication Number WO 94/05659. A particularly preferred form of Compound A is the maleate salt.

Suitable pharmaceutically acceptable solvates include hydrates.

The present invention further provides the use of Compound A, or a pharmaceutically acceptable salt or solvate thereof, and a pharmaceutically acceptable glyceride-based waxy mixture of materials in the manufacture of a controlled release oral dosage form for treating the Disorders of the Invention.

As indicated above, the oral dosage form of the present invention is considered to be suitable for once daily administration and during use is indicated to provide a therapeutic effect over an extended period of time, such as up to 24 hours, for example, up to 12, 14, 16, 18, 20 and 24 hours, per unit dose.

In the treatment and/or prophylaxis of the above-mentioned conditions, the oral dosage forms of this invention may be taken in amounts so as to provide Compound A in suitable doses, such as those disclosed in EP 0,306,228, WO 94/05659 or WO 98/55122. For example, a suitable dosage range is up to 12 mg, for example, 1 to 12 mg. Thus, suitable dosage forms comprise 1, 2, 3, 4, 5, 6, 7, 8, 9, 10, 11 or 12 mg of Compound A or a pharmaceutically acceptable salt or solvate thereof.

In one particular aspect, the oral dosage form comprises 2 to 12 mg of Compound A.

Suitably the oral dosage form comprises 2, 3, 4, 5, 6, 7, 8, 9, 10, 11 or 12 mg of Compound A.

Particularly, the oral dosage form comprises 2 to 4, 4 to 8 or 8 to 12 mg of Compound A.

Particularly, the oral dosage form comprises 2 to 4 mg of Compound A.

Particularly, the oral dosage form comprises 4 to 8 mg of Compound A.

Particularly, the oral dosage form comprises 8 to 12 mg of Compound A.

Preferably, the oral dosage form comprises 2 mg of Compound A.

Preferably, the oral dosage form comprises 4 mg of Compound A.

Preferably, the oral dosage form comprises 8 mg of Compound A.

Most preferably the oral dosage forms are formulated to deliver a dose of 8 mg of Compound A (as the free base) in a sustained release as a once a day dose.

Where the oral dosage form of the invention comprises a first and a second composition arranged as discrete layers, the amount of Compound A or a pharmaceutically acceptable salt or solvate thereof present in each of the layers may be varied in accordance with the desired dissolution profile.

For example, where the oral dosage form comprises 8 mg of Compound A or a pharmaceutically acceptable salt or solvate thereof, the oral dosage form suitably comprises a layer comprising 1 mg of Compound A or a pharmaceutically salt or solvate thereof, and a layer comprising 7 mg of Compound A or a pharmaceutically salt or solvate thereof. Alternatively, the oral dosage form may comprise a layer comprising 4 mg of Compound A or a pharmaceutically salt or solvate thereof, and a layer comprising 4 mg of Compound A or a pharmaceutically salt or solvate thereof. More suitably, the oral dosage form comprises a layer comprising 2 mg of Compound A or a pharmaceutically salt or solvate thereof, and a layer comprising 6 mg of Compound A or a pharmaceutically salt or solvate thereof. Preferably, the oral dosage form comprises a layer comprising 3 mg of Compound A or a pharmaceutically salt or solvate thereof, and a layer comprising 5 mg of Compound A or a pharmaceutically salt or solvate thereof.

Where the oral dosage form comprises 2 mg of Compound A or a pharmaceutically acceptable salt or solvate thereof, the oral dosage form suitably comprises a layer comprising 0.75 mg of Compound A or a pharmaceutically salt or solvate thereof, and a layer comprising 1.25 mg of Compound A or a pharmaceutically salt or solvate thereof.

Where the oral dosage form comprises 4 mg of Compound A or a pharmaceutically acceptable salt or solvate thereof, the oral dosage form suitably comprises a layer comprising 1.5 mg of Compound A or a pharmaceutically salt or solvate thereof, and a layer comprising 2.5 mg of Compound A or a pharmaceutically salt or solvate thereof.

For the avoidance of doubt, when reference is made herein to scalar amounts, including mg amounts, of Compound A in a pharmaceutically acceptable form, the scalar amount referred to is made in respect of Compound (I) per se. For example, 2 mg of Compound (I) in the form of the maleate salt is that amount of maleate salt, which contains 2 mg of Compound (I).

All publications, including but not limited to patents and patent applications, cited in this specification are herein incorporated by reference as if each individual publication were specifically and individually indicated to be incorporated by reference herein as though fully set forth.

No adverse toxicological effects are indicated in the above mentioned treatments for the oral dosage forms of the invention.

The invention is illustrated by the following Examples.

### EXAMPLE 1

Gelucire 50/13 (Gattefosse) and Precirol ATO5 (Gattefosse) were melt blended at 70° C. The temperature of the blend was allowed to decrease to between 52 and 57° C. Compound (A) as the maleate (GlaxoSmithKline) was added to the molten blend, so that the resultant mixture contained the three components in the proportions

| | % w/w |
|---|---|
| Compound (A) Maleate | 4 |
| Gelucire 50/13 (wax) | 46 |
| Precirol ATO5 (wax) | 50 |

The molten mixture was filled into rubber tablet moulds and allowed to cool, to give tablets of total weight 269 mg, each containing 8 mg of Compound (A) (measured as the free base). Tablets were coated with a solution of Opadry 2, to a 6% weight gain.

The moulded and coated tablets were then heated for 48 hours at 40° C., to improve the physical stability, and the reproducibility of dissolution release rates.

### EXAMPLE 2

Gelucire 50/13 (Gattefosse) and Precirol ATO5 (Gattefosse) were melt blended at 70° C. The temperature of the blend was allowed to decrease to between 52 and 57° C. Compound (A) Maleate (GlaxoSmithKline) was added to the molten blend, so that the resultant mixture contained the three components in the proportions

| | % w/w |
|---|---|
| Compound (A) Maleate | 2.65 |
| Gelucire 50/13 (wax) | 37.35 |
| Precirol ATO5 (wax) | 60 |

The molten mixture was filled into rubber tablet moulds and allowed to cool, to give tablets of total weight 400 mg, each containing 8 mg of Compound (A) (measured as the free base). Tablets were coated with a solution of Opadry 2, to a 6% weight gain.

The moulded and coated tablets were then heated for 48 hours at 40° C., to improve the physical stability, and the reproducibility of dissolution release rates.

### EXAMPLE 3

Gelucire 50/13 (Gattefosse) and Precirol ATO5 (Gattefosse) were melt blended at 70° C. The temperature of the blend was allowed to decrease to between 55 and 60° C. Compound (A) Maleate (GlaxoSmithKline) was added to the molten blend, so that the resultant mixture contained the three components in the proportions

| | % w/w |
|---|---|
| Compound (A) Maleate | 4 |
| Gelucire 50/13 (wax) | 46 |
| Precirol ATO5 (wax) | 50 |

The molten mixture was filled into capsules and allowed to cool. Each capsule contained 8 mg of rosiglitazone (measured as the free base).

Dissolution Tests

Dissolution rates for the formulations of Examples 1 and 2 were measured starting at pH 1.5 with an adjustment to pH 6.8 after 4 hours, as an assumed time for residence in the fed stomach before emptying into the intestines. The medium for this dissolution test is initially an aqueous solution of sodium chloride and hydrochloric acid, pH 1.5 to mimic the pH found in the stomach environment. This medium is then titrated to pH 6.8 by the addition of aqueous sodium dodecyl sulfate and an aqueous solution of sodium acetate and tris(hydroxymethyl)methylamine after 4 hours to mimic the pH found in the intestine. The results are plotted in FIG. 1. The formulation of Example 2 gave a slower release of rosiglitazone than the tablet of Example 1, by virtue of the increased amount of Precirol ATO 5, giving a more hydrophobic character to the matrix, and because of the increased tablet size.

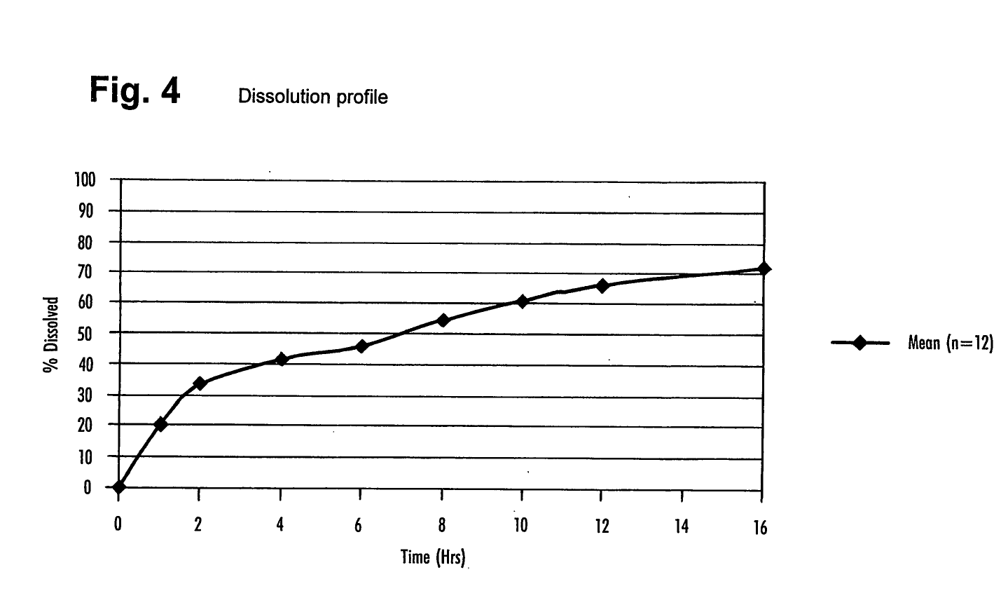

Dissolution rates for the formulation of Example 3 were measured starting at pH 1.5 with an adjustment to pH 7.4 after 2 hours, as an assumed time for residence in the fasted stomach before emptying into the intestines. The medium for this dissolution test is initially an aqueous solution of sodium chloride and hydrochloric acid, pH 1.5 to mimic the pH found in the stomach environment. This medium is then titrated to pH 7.4 by the addition of aqueous sodium dodecyl sulfate and an aqueous solution of sodium acetate and tris(hydroxymethyl)methylamine after 2 hours to mimic the pH found in the intestine. The results are shown in FIG. 4.

1. A controlled release oral dosage form comprising 5-[4-[2-(N -methyl-N-(2 pyridyl)amino)ethoxy]benzyl]thiazolidine-2,4-dione or a pharmaceutically acceptable salt or solvate thereof, dispersed in a carrier comprising a pharmaceutically acceptable waxy mixture of glyceride-based materials, having an HLB value of 4 to 12, and an average melting point in the range of 50 to 55° C.

2. A controlled release oral dosage form comprising 5-[4-[2-(N -methyl-N-(2 pyridyl)amino)ethoxy]benzyl]thiazolidine-2,4-dione or a pharmaceutically acceptable salt or solvate thereof, dispersed in a carrier comprising a mixture of
(a) a pharmaceutically acceptable waxy mixture of glyceride-based materials having an HLB value of greater than 12 and an average melting point in the range of 50 to 55° C., and an amount of
(b) a pharmaceutically acceptable fatty acid glyceride or glyceride mixture having an HLB value less than the HLB value of component (a) and an average melting point in the range of 50 to 55° C.,
such that the carrier as a whole has an HLB value of 4 to 12.

3. A controlled release oral dosage comprising a first composition and a second composition, each composition comprising 5-[4-[2-(N-methyl-N-(2 pyridyl)amino)ethoxy]benzyl]thiazolidine-2,4-dione or a pharmaceutically acceptable salt or solvate thereof and a pharmaceutically acceptable carrier therefore, wherein:
(a) the carrier of the first composition comprises a pharmaceutically acceptable waxy mixture of glyceride-based materials having a range of HLB values of 4 to 12 and an average melting point in the range of 50 to 55° C.; and
(b) the carrier of the second composition comprises one or more pharmaceutically acceptable glyceride-based materials having a higher HLB value and/or lower average melting point than the carrier of the first composition.

4. An oral dosage form according to claim 3, in which the first and second compositions are arranged to release 5-[4-[2-(N-methyl-N-(2 pyridyl)amino)ethoxy]benzyl]thiazolidine-2,4-dione or a pharmaceutically acceptable salt or solvate thereof, at differing release rates on administration such that the rate of release of the drug from the dosage form is substantially independent of pH.

5. An oral dosage form according to claim 4, in which the release rate of 5-[4-[2-(N-methyl-N-(2 pyridyl)amino)ethoxy]benzyl]thiazolidine-2,4-dione or a pharmaceutically acceptable salt or solvate thereof from the second composition is substantially greater than from the first composition.

6. An oral dosage form according to claim 5, in which the second composition is an immediate release composition.

7. An oral dosage form according to claim 5, in which the first composition is a controlled release composition.

8. An oral dosage form according to claim 1, in which the pharmaceutically acceptable waxy mixture of glyceride-based materials is waxy material obtainable by an alcoholysis/esterification reaction between a vegetable oil and a polyethylene glycol.

9. An oral dosage form according to claim 8, in which the vegetable oil is a hydrogenated oil.

10. An oral dosage form according to claim 9, in which the vegetable oil is hydrogenated palm oil.

11. An oral dosage form according to claim 2, in which the fatty acids of the glyceride are predominantly palmitic and stearic acids.

12. An oral dosage form according to claim 1, in which the carrier and 5-[4-[2-(N-methyl-N-(2 pyridyl)amino)ethoxy]benzyl]thiazolidine-2,4-dione or a pharmaceutically acceptable salt or solvate thereof are moulded to form a tablet.

13. An oral dosage form according to claim 1, in which the carrier and 5-[4-[2-(N-methyl-N-(2 pyridyl)amino)ethoxy]benzyl]thiazolidine-2,4-dione or a pharmaceutically acceptable salt or solvate thereof are filled into capsule shells to form swallow capsules.

14. A method for the treatment and/or prophylaxis of diabetes mellitus, conditions associated with diabetes mellitus and certain complications thereof, osteoporosis, Alzheimer's Disease, psoriasis, asthma and metabolic syndrome, which comprises administering an effective amount of a controlled release oral dosage form as claimed in claim 1 to a human or non-human mammal in need thereof.

15. (canceled)

16. A method of preparing a controlled release oral dosage form according to claim 1 which comprises dispersing 5-[4-[2-(N-methyl-N-(2 pyridyl)amino)ethoxy]benzyl]thiazolidine-2,4-dione or a pharmaceutically acceptable salt or solvate thereof, in a molten carrier comprising a pharmaceutically acceptable waxy mixture of glyceride-based materials, having an HLB value of 4 to 12, and an average melting point in the range of 50 to 55° C., filling the molten mixture into tablet moulds or capsule shells, allowing the carrier to solidify, and optionally thereafter maintaining the solidified dosage form at a temperature of at least 40° C., but below the melting point of the carrier, for a time sufficient to allow the carrier to achieve a stable polymorphic form.

17. A method of preparing a controlled release oral dosage form according to claim 2 which comprises dispersing 5-[4-[2-(N-methyl-N-(2 pyridyl)amino)ethoxy]benzyl]thiazolidine-2,4-dione or a pharmaceutically acceptable salt or solvate thereof, in a molten carrier comprising a mixture of
(a) a pharmaceutically acceptable waxy mixture of glyceride-based materials having an HLB value of greater than 12 and an average melting point in the range of 50 to 55° C., and an amount of
(b) a pharmaceutically acceptable fatty acid glyceride or glyceride mixture having an HLB value less than the HLB value of component (a) and an average melting point in the range of 50 to 55° C.,
such that the carrier as a whole has an HLB value of 4 to 12,
filling the molten mixture into tablet moulds or capsule shells, allowing the carrier to solidify, and maintaining the solidified dosage form at a temperature of at least 40° C., but below the melting point of the carrier, for a time sufficient to allow the carrier to achieve a stable polymorphic form.
